(19) United States
(12) Patent Application Publication
(10) Pub. No.: US 2021/0338173 A1
(43) Pub. Date: Nov. 4, 2021

(54) SYSTEM AND METHOD FOR PREDICTING EXERTIONAL HEAT STROKE WITH A WORN SENSOR

(71) Applicant: Massachusetts Institute of Technology, Cambridge, MA (US)

(72) Inventors: Jeffrey Palmer, Westford, MA (US); Brian Telfer, Lincoln, MA (US); James Williamson, Arlington, MA (US); Lara Weed, East Kingston, NH (US); Mark Buller, Douglas, MA (US); Rebecca Fellin, Natick, MA (US); Joseph Seay, Marlborough, MA (US)

(73) Assignees: Massachusetts Institute of Technology, Cambridge, MA (US); U.S. Army Research Institute of Environmental Medicine, Natick, MA (US)

(21) Appl. No.: 16/910,614

(22) Filed: Jun. 24, 2020

Related U.S. Application Data
(60) Provisional application No. 63/019,147, filed on May 1, 2020.

(51) Int. Cl. A61B 5/00; A61B 5/0205; A61B 5/11

(52) U.S. Cl. CPC A61B 5/7275; A61B 5/02438; A61B 5/1123; A61B 5/02055

(57) ABSTRACT

A heat response monitor, comprises an accelerometer, a core temperature sensor, an estimation device, and an enabler. The estimation device uses accelerometry-based functionality to provide a gait-based heat stroke risk score, and the estimation device uses an estimated core temperature of a wearer of the core temperature sensor, to provide an estimated core temperature-based heat stroke risk score. The gait-based heat stroke risk score and the estimated core temperature-based heat stroke risk score are used to determine if a wearer of the heat response monitor is in risk of heat injury.

### CROSS-REFERENCE TO RELATED APPLICATIONS

This application claims the benefit of U.S. Provisional Patent Application Ser. No. 63/019,147, filed May 1, 2020, entitled “System and Method to Predict Exertional Heat Stroke from Torso-Worn Sensor,” which is incorporated by reference herein in its entirety.

### GOVERNMENT SUPPORT STATEMENT

This invention was made with Government support under Grant No. FA8702-15-D-0001 awarded by the U.S. Air Force. The Government has certain rights in the invention.

### FIELD OF THE INVENTION

The present invention generally relates to detection of heat related illness and, more particularly, to systems and methods that provide early detection of the potential for heat stroke.

### BACKGROUND OF THE INVENTION

Heat stroke, the most serious form of heat injury, can result in death or permanent disability. People most susceptible to the effects of heat injury include those who consistently work hard in the heat, including, but not limited to, military service-members, construction workers, firefighters and other first responders, athletes, miners, agricultural workers, and maintenance workers in hot, enclosed spaces. The number of exertional heat strokes among U.S. active-duty service members almost doubled between 2008 and 2018, with 578 reported cases in 2018. The seriousness of the problem in the military has even been the subject of news articles and videos. Heat injuries are also recognized as a leading cause of death and disability in U.S. high school and college athletes, and more broadly, 5,946 people were treated in U.S. emergency departments each year for heat injuries during athletic or recreational activities. Outside of the U.S., hundreds of construction workers are dying each year in Qatar alone.

Wet bulb globe temperature and related indices based on ambient temperature and humidity are presently used to assess group-average risk of heat injury, but because of significant variations in human physiology, these indices are inadequate to predict an individual's heat injury risk. Insight into an individual's physiological state can be gained through monitoring the rise of core body temperature, because one of the main signs of heat stroke is considered to be a core body temperature of 104° F. or above. However, the gold standard methods for measuring core body temperature—rectal probes or ingestible sensors—are not acceptable or practical for routine use. In addition, high core body temperature does not always lead to heat stroke; for example, well-trained runners have been seen to reach and maintain 104° F. with no ill effects in cool ambient temperatures. Heat injuries can also occur at lower core body temperature (e.g., in individuals who are not heat acclimatized or who have preexisting health conditions). The most practical method for noninvasively estimating core temperature is an algorithm developed by the United Stated Army Research Institute of Environmental Medicine (USARIEM) that operates on heart rate. The U.S. Army has licensed this technology to Hidalgo and Zephyr. These companies have integrated the estimated core temperature in their wearable sensors. However, even when using a gold standard core body temperature method, core temperature alone does not provide a high enough prediction specificity. As would be expected, when the core temperature estimation algorithm is utilized alone with a simple threshold an unacceptably high false alarm rate occurs. For effectiveness, a method with high true positive rate and low false alarm rate is required.

### SUMMARY OF THE INVENTION

Embodiments of the present invention provide systems and methods for predicting exertional heat stroke with a worn sensor. Briefly described, the present invention utilizes 3 axis accelerometry to determine a gait stability index and heart rate to estimate core body temperature. In combination these two measures allow for exertional heat stroke to be predicted with high specificity and sensitivity, well in advance of collapse. The method for predicting exertional heat stroke with a worn sensor, comprises the steps of: detecting individual steps in time domain from accelerometry, as data is received from an accelerometer, where accelerometry data comprises a time series of 3-axis accelerations; classifying each step as a walking or running step; classifying frames of steps as walking frames or running frames; for frames classified as either walking frames or running frames, computing an autocorrelation of time series data in each acceleration axis x(t), y(t), and z(t) of the frame; computing average pairwise sample distance in each acceleration axis; computing change in movement variability features, relative to recent history of feature statistics; and applying a threshold to a fused risk score, which is a combination of accelerometry-based risk score and estimated core temperature based risk score to predict heat injury.

The heat response monitor comprises an accelerometer, a core temperature sensor, an estimation device, and an enabler. The estimation device uses accelerometry-based functionality to provide a gait-based heat stroke risk score, and the estimation device uses an estimated core temperature of a wearer of the core temperature sensor, to provide an estimated core temperature-based heat stroke risk score. The gait-based heat stroke risk score and the estimated core temperature-based heat stroke risk score are used to determine if a wearer of the heat response monitor is in risk of heat injury.

Other systems, methods and features of the present invention will be or become apparent to one having ordinary skill in the art upon examining the following drawings and detailed description. It is intended that all such additional systems, methods, and features be included in this description, be within the scope of the present invention and protected by the accompanying claims.

### BRIEF DESCRIPTION OF THE DRAWINGS

The accompanying drawings are included to provide a further understanding of the invention, and are incorporated in and constitute a part of this specification. The components in the drawings are not necessarily to scale, emphasis instead being placed upon clearly illustrating the principles of the present invention. The drawings illustrate embodiments of the invention and, together with the description, serve to explain the principals of the invention.

FIG. 1 is a schematic diagram illustrating part of the present system for early predicting of exertional heat stroke in an individual with a high degree of accuracy.

FIG. 2 is an image demonstrating wearing of the heat response monitor of the first exemplary embodiment of the invention, in accordance with one wearing position.

FIG. 3 is a flowchart of an exemplary embodiment of a method performed by the estimation device to determine if a user has a decrease in gait stability.

FIG. 4 is a schematic diagram illustrating embodiments in which the estimation device functionality and the enabler functionality are provided within a computer.

### DETAILED DESCRIPTION

The present system and method uses the combination of physiologic and neurological insight, including movement and motion of an individual, physiological and neurological response of the individual's body, and estimated core temperature to provide real-time, early detection of the potential for a person to have heatstroke. The system and method is based on the observation that people often exhibit ataxia, or decreased gait stability, in the few minutes prior to a heat injury. By providing highly accurate, real-time, early detection, steps can be taken to minimize, and even prevent heat stroke.

The present system and method incorporates and works with a wearable sensor, in order to allow noninvasive, nonobtrusive, accurate monitoring of people who are active and possibly encumbered by wearing protective equipment.

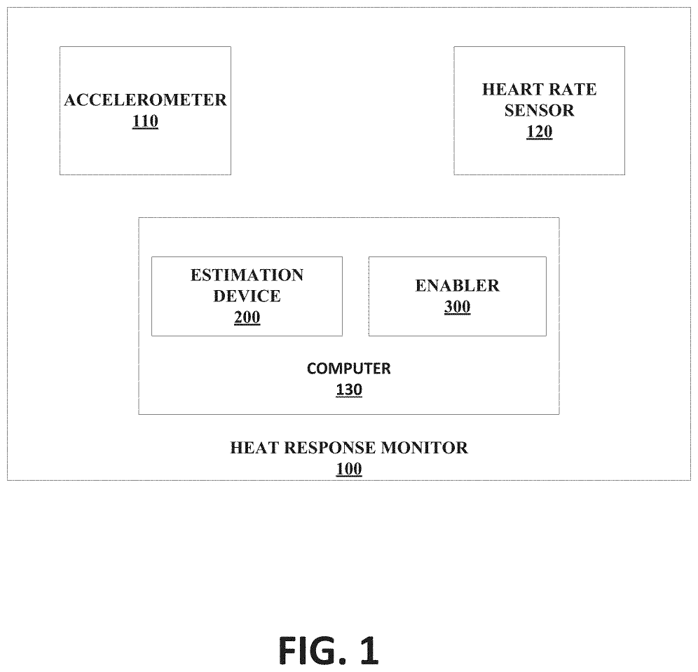

FIG. 1 is a schematic diagram illustrating part of the present system for early predicting of exertional heat stroke in an individual with a high degree of accuracy, so as to allow for time to change actions, to seek medical attention, or to have medical support deployed to the individual in advance of heat stroke. For example, if all members of a football team were using the present system and method, the system and method would alert responsible parties well in advance of a possible heat stroke, yet with a high degree of accuracy, so as to allow those football team members in need using the present system to change their actions or receive medical attention in advance of damage, as well as minimizing false alerts.

Multiple embodiments of the present invention may be provided. In accordance with a first exemplary embodiment of the invention, a single unit may be provided, referred to herein as a heat response monitor 100. In accordance with the first exemplary embodiment of the invention, the heat response monitor 100, which has multiple portions therein, as will be described herein, is capable of obtaining all necessary biological information of the person wearing the monitor 100 for determining if current condition of the person wearing the monitor 100 shows that they are highly likely to suffer a heat stroke. In addition, in the first exemplary embodiment of the invention, functionality for analyzing data obtained by the heat response monitor 100 to determine if there is a likelihood of heat stroke in the individual wearing the device is also located within the heat response monitor 100, as described herein. Such functionality may be defined within components of a computer, or other form of logic, examples of which are described herein.

Referring to FIG. 1 the heat response monitor 100 of the first exemplary embodiment of the invention contains an accelerometer 110, a heart rate sensor 120, and a computer 130 having an estimation device 200 and an enabler 300 therein. It is noted that the accelerometer 110 and heart rate sensor 120 may instead be a single sensor for detecting both heart rate and movement accelerations. Since heart rate sensors and acceleration sensors (accelerometers) are known to those having ordinary skill in the art, further description of the same is not provided herein.

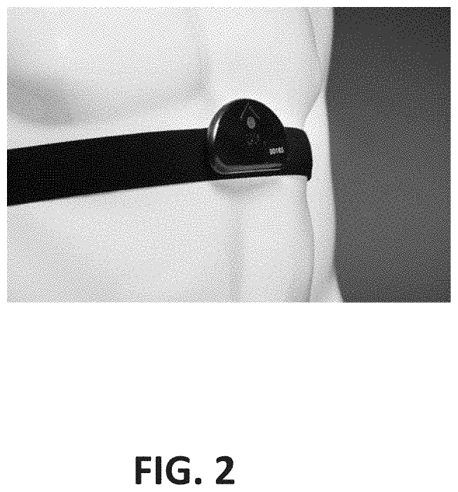

FIG. 2 is an image demonstrating wearing of the heat response monitor 100 of the first exemplary embodiment of the invention. While the image demonstrates wearing of the monitor 100 around the torso, it should be noted that the monitor 100 may instead be located around the arm, shoulder, or even wrist. In fact, the monitor 100 may be incorporated into a watch, where all components are located on the wrist watch. Such a wrist watch embodiment allows the present monitor 100 to be incorporated into a wrist watch having other functionality therein and adds to convenience of use.

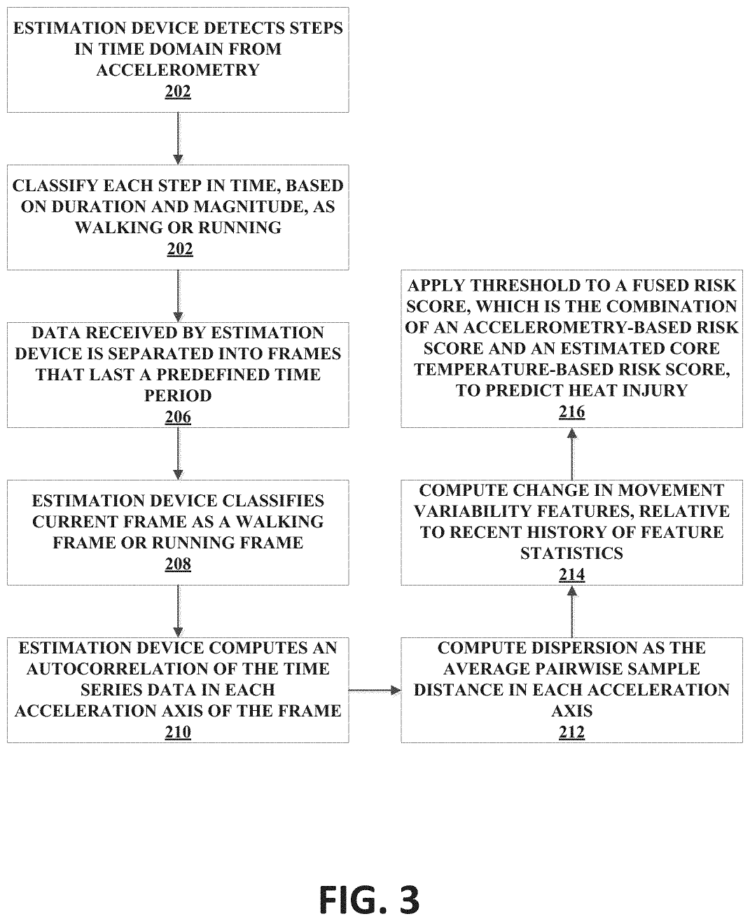

Returning back to FIG. 1, the accelerometer 110 keeps track of movement of the user, as is known by those having ordinary skill in the art. In the case of wearing the monitor 100 on the torso, as shown by FIG. 2, the acceleration data provided by the accelerometer 110 is torso acceleration. The estimation device 200 receives data time series acceleration data from the accelerometer 110 and determines if the user has a decrease in gait stability. To make this determination, the estimation device 200 performs the steps as shown in the flowchart of FIG. 3.

FIG. 3 is a flowchart of an exemplary embodiment of a method performed by the estimation device 200 to determine if a user has a decrease in gait stability. It should be noted that any process descriptions or blocks in flowcharts should be understood as representing modules, segments, portions of code, or steps that include one or more instructions for implementing specific logical functions in the process, and alternative implementations are included within the scope of the present invention in which functions may be executed out of order from that shown or discussed, including substantially concurrently or in reverse order, depending on the functionality involved, as would be understood by those reasonably skilled in the art of the present invention.

As shown by block 202, the estimation device 200 first detects individual steps in time domain from accelerometry, as data is received from the accelerometer 110. Accelerometry data consists of a time series of 3-axis accelerations, x(t)={x_1(t),x_2(t),x_3(t)} from the accelerometer 110, with x_1=vertical, x_2=longitudinal, and x_3=horizontal, and sampling frequency being fs=128 Hz. Specifically, two successive peaks in the vertical axis, x_1(t), acceleration are detected. The vertical signal may be smoothed in the time domain with a 6th-order low-pass Butterworth filter with frequency cutoff of, for example, 6 Hz. Local negative peaks are detected in the smoothed signal with a minimum peak prominence of, for example, 0.3 g. Each pair of successive peaks with an interpeak interval between, for example, 0.1713 and 1.5 s represent a detected step, with the peaks demarcating the start and stop times of the step.

If the time duration between these peaks falls within a specified range and the standard deviation of the acceleration magnitude during this duration falls within a specified range, then the step is detected as a walking or running step. For exemplary purposes only, and not provided for limiting purposes, these ranges may be as follows. Walking duration: 0.4 to 0.8 seconds. Running duration: 0.24 to 0.5 seconds. Walking magnitude standard deviation: 0.25 to 1.0 g. Running magnitude standard deviation: 0.352 to 2.8 g.

Each step in time is classified by the estimation device 200 as a walking or running step (block 204). The classification is based on step duration and standard deviation of acceleration magnitude, which is computed over the duration of the step. The accelerometer provides tri-axial accelerations at a certain data rate (for example, 128 Hz or 128 data points per second). The duration is the elapsed time between peaks in these acceleration data. The “magnitude” is the standard deviation of the individual magnitudes. The individual magnitudes at a given time point t are: m(t)=sqrt(x(t)\hat{ }2+y(t)\hat{ }2+z(t)\hat{ }2) where x(t), y(t), z(t) are acceleration in the three axes at time t.

Data received thus far by the estimation device 200 is separated into frames that last a predefined time period (block 206). For example, a frame may be five seconds long, although a frame need not be as long or as short as five seconds. As shown by block 208, the estimation device 200 then classifies the current frame as a walking frame or a running frame by checking to see if a particular frame includes consistent walking or running. As an example, the estimation device 200 can classify a frame as walking if all steps in the frame were classified as walking, and similarly for running. More generally, it could do this if a sufficiently high fraction of steps in the frame were classified as walking or running. The classification of each frame is stored within the heat response monitor 100, for instance, within a database of the computer 130.

As shown by block 210, for frames classified as either walking frames or running frames, and therefore, including consistent walking or consistent running so as to allow that frame to be identified as a walking frame or a running frame, the estimation device 200 computes an autocorrelation of the time series data in each acceleration axis, x(t), y(t) and z(t) of the frame. As is known by those having ordinary skill in the art, autocorrelation is also known as serial correlation, which is the correlation of a signal with a delayed copy of itself as a function of delay.

Let n be the index for the current data frame. Once frame n is classified as walking or running, then autocorrelation peak height features are extracted, which represent the level of regularity of steps within the frame. Autocorrelations of the 5 s acceleration signals in each axis are computed. The first autocorrelation peak represents the average step time duration (peak time delay) and level of step consistency (peak height). The range of allowed time delays depends on the frame gait type. Autocorrelation peaks are only valid for time delays>than the smallest time delay in which the autocorrelation is negative. Walking frames require the peak time delay to be <0.86 s, and running frames the peak time delay to be between <0.47 s. The height of the vertical autocorrelation peak, H_1(n), is required to be suprathreshold, H_1(n)>H_min, where H_min=0.4. If there is no vertical autocorrelation peak that fits the above criteria, then the frame is reclassified as another frame (neither walking or running), and further feature processing is discontinued. This is done because a low vertical autocorrelation peak indicates a lack of periodicity that is expected of regular gait. If the vertical peak is valid according to the above criteria, then autocorrelation peak heights are computed independently in the other two axes as well: H_2(n) and H_3(n).

After the autocorrelation is computed in each acceleration axis, the extracted feature is the height of the first autocorrelation peak in each acceleration axis. The value of the first autocorrelation peak at >0 time delay is selected as a feature for indicating the consistency of the acceleration patterns comprising each step within the frame, providing an index of gait stability for both walking and running.

A separate index of incoordination is dispersion. Gait signals are quasiperiodic and acceleration signals in nearby steps will typically tend to repeat the same patterns. Dispersion indicates the extent to which this does not happen. The more the accelerometry values from nearby (in time) steps differ from each other at similar points in the gait cycle, the higher the dispersion will be. As shown by block 212, dispersion is computed as the average pairwise sample distance in each acceleration axis. Dispersion is a measure of the average distance between normalized accelerations across a frame of data. First, the acceleration values in frame n are z-scored into standard units in each axis, yielding xz_i(t). Next, outlier points are removed from analysis, as these seem to degrade the usefulness of the dispersion feature. Let V(n) be the set of valid (i.e., non-outlier) time points in frame n, defined by excluding points greater than two standard deviations in any of the three axes:

V(n)=t such that max_i(xz_i(t))<2 (eq. 1)

Next, average distances between all pairs of valid values in each axis are computed. For computational efficiency, L1 distances are used. The dispersion (D) in axis i is thus

D_i(n)=1/n\sum_{t1 in V(n), t2 in V(n)}|xz_i(t1)−xz_i(t2). (eq. 2)

The dispersion is the sum of absolute pairwise differences in each axis, x(t), y(t), and z(t). The dispersion in a single axis, x(t), is the average absolute difference between all pairs of values in that frame: mean_t,t′ (|x(t)−x(t′)|). It is noted that the mean is computed over all pairs of values given by t and t′, where t and t′ are time points within that frame. In the current implementation, dispersion is a positive index of gait instability in the running frames. This is based on empirical observation that increasing dispersion is a predictor of heat illness during running. Dispersion is also used as a negative index in the vertical axis in the walking frames, as it has empirically been observed that reduction of vertical dispersion is predictive of heat illness during walking.

As shown by block 214, the estimation device 200 then computes change in movement variability features, relative to recent history of feature statistics by using a recursive filter that normalizes (z-scores) and smooths the features over time, as explained hereinafter. The term “recent history” for the variability features is a function of the number of valid frames that have been processed and gaps between the frames, and therefore is a deterministic function that is based on equations 3 through 8 as described hereinafter. For each feature, two values are stored, a first being the mean of the feature, and a second being the mean of the feature squared. For each gait class (walking, running) a weight is stored, which essentially represents the frame count. The normalization results in the gait stability indices having units of standard deviations from a baseline.

A risk score is updated for each feature by use of filtering, after each data frame is processed. The filtering, which can be performed by an algorithm or otherwise, is designed to detect positive changes in a feature (toward a more erratic gait) with respect to the individual's own recent distribution of feature values. Thus, it detects change in a feature that is calibrated by the individual's recent mean and standard of that feature. The filter consists of three steps. First, it updates sufficient statistics, which are the first and second moment of the feature. Second, it uses these statistics to update on-line z-scoring of the feature, which maps the current feature value into standard units (zero mean, unit variance) with respect to its history. Third, it does smoothing of the z-scored feature value.

Below is a description of methods for performing feature change detection based on z-scoring. Let f(n) be a gait accelerometry variability feature at frame n. For a walking frame, in acceleration axis i, the correlation height feature is f(n). f(n) could be the negative of the height of an autocorrelation peak in one of the acceleration axes, or it could be the dispersion feature in one of the three acceleration axes. For purposes of computation, f(n)=−H_i(n)\hat{ }{w}, and the dispersion feature is f(n)=−D_i(n)\hat{ }{w}. For a running frame, in acceleration axis i, the correlation height feature is f(n)=−H_i(n)\hat{ }{r}, and the dispersion feature is f(n)=D_i(n)\hat{ }{r}. Notice that negative dispersion is an indicator of risk for walking, whereas positive dispersion is an indicator of risk for running.

The statistical variables f1(n) and f2(n) are updated to represent the first moment and second moment of f(n).

Initialization: when n=1,

f1(n)=f(n) (eq. 3)

f2(n)=f(n)\hat{ }2. (eq. 4)

Updating: when n>1,

f1(n)=1/w(n) f(n)+(1−1/w(n)) f1(n−1) (eq. 5)

f2(n)=1/w(n) f(n)\hat{ }2+(1−1/w(n)) f2(n−1) (eq. 6)

The updating rate depends on a variable w(n) that increases with frame count but also decays over time in the absence of frames, thereby forgetting the past and resetting the on-line process of estimating feature changes.
Initialization: when n=1, w(n)=1.
Updating: when n>1, w(n)=1+w(n−1)*exp(−gap_coeff*gap_length)
gap_length is the number of frames since the last time a valid frame (of walking or running) has been processed. For exemplary purposes, gap_coeff is currently set to 0.005. This number determines how quickly the information from previous frames is discarded across a gap in which there are no frames processed. For example, a large gap_coeff value could cause the statistics to be almost completely reset after a short gap of only minutes, whereas a small gap_coeff value could cause the statistics to remain stable across a gap of several hours. The choice of how to set this depends on how stable a person's gait is expected to be, and therefore how much the statistics from previous gait frames can be trusted as a baseline, compared to which gait deviations will be used to detect risk of heat illness.

The following explains how z-scoring is performed. The first and second moments, f1(n) and f2(n), are used to do z-scoring to map the feature into standard units (zero mean, unit standard deviation):

fz(n)=(f(n)−f1(n))/sqrt(f2(n)−f1(n)\hat{ }2+1/w(n)). (eq. 7)

The extra term in the denominator, w(n), prevents fz(n) from fluctuating too wildly when w(n) is small.

Finally, filtering is performed to produce a smoothed heat risk score, fzs(n). Smoothing is done to reduce local fluctuations in the values of f(n), which can reduce the accuracy of heat risk detections by raising the level of false alarms for any given detection threshold.

fzs(n)=alpha fz(n)+(1−alpha)fzs(n−1) (eq. 8)

fzs (n) is produced for each f(n) feature, i.e., for each accelerometry axis and for each feature type (i.e., for autocorrelation height in each axis, and for dispersion in each axis). These smoothed risk scores are denoted as: H_risk_i(n)\hat{ }{w}, H_risk_i(n)\hat{ }{r}, D_risk_i(n)\hat{ }{w} and D_risk_i(n)\hat{ }{r}, for the three acceleration axes, i={1, 2, 3}. These features are combined by summing them. Separate risk scores are computed for walking and running gate

Gait_risk(n)\hat{ }{w}=\sum_i=1:3 H_risk_i(n)\hat{ }{w}+D_risk_1(n)\hat{ }{w} (eq. 9)

Gait_risk(n)\hat{ }{r}=\sum_i=1:3 H_risk_i(n)\hat{ }{r}+\sum_i=1:3 D_risk_i(n)\hat{ }{r} (eq. 10)

Gait_risk(n)=max(Gait_risk(n)\hat{ }{w}, Gait_risk(n)\hat{ }{r}) (eq. 11)

Heat risk scores may then be computed from estimated core temperature. Estimated core temperature, ECT(n), is computed from heart rate, and used as the basis for a baseline risk score based on temperature. This is done using a Gaussian log-likelihood function when ECT(n)<ECT_max, as shown by equation 11.

ECT_risk(n)=0, when ECT>=ECT_max

−0.5(ECT(n)−ECT_max)\hat{ }2/ECT_sigma\hat{ }2 otherwise (eq. 12)

ECT_max=41 with ECT_sigma=1. For exemplary purposes, ECT_max is selected to be 41 because this is the highest plausible value that ECT(n) is expected to reach. If ECT_max were set lower, then the ECT_risk(n) score would reach its maximum value of 0 at a lower values of ECT(n). In addition, ECT sigma is selected to be 1 because this is a reasonable representation of variability in ECT(n) estimates across subjects, given that they have the same true core temperature. ECT_sigma determines how rapidly ECT_risk(n) drops as ECT falls below 41.

Fused risk scores for walking and running are produced by adding the Gait_risk and ECT_risk scores:

Fused_Risk(n)=Gait_risk(n)+ECT_risk(n) (eq. 13)

The accelerometry-based functionality, as described above, which could be provided by software associated with an algorithm, firmware, logic within a chip, or one or more of many different ways, provides a gait-based heat stroke risk score. Similarly, the estimated core temperature determined by the heart rate sensor 120, provides a separate estimated core temperature-based heat stroke risk score.

The accelerometry-based risk score and the ECT-based risk score are added together to produce a fused risk score as described in equation (eq. 13). It is noted that core temperature may be determined through use of a heart rate monitor alone, an example of which is demonstrated in US publication number 20170238811, which is incorporated herein by reference in its entirety. Alternatively, the core temperature may be determined by use of another known device to one having ordinary skill in the art.

A threshold is then applied to the fused risk score in equation 13 in order to predict heat injury (block 216). The particular threshold one chooses is based on how one wants to balance false positive predictions with false negative predictions. The threshold can be varied to trade off detection and false alarm probability and advanced warning time.

The enabler 300 performs the function of providing a total risk score which continually quantifies incoordination as a person walks or runs, based on the monitor 100 worn accelerometry. The risk score is individualized in terms of its accelerometry-based features, in that the levels of those features are compared to their statistical distribution as measured in previous data frames from the same individual. If the index exceeds a threshold, then a warning is issued to the user as a sound, vibration, or using any other output that would allow the individual wearing the monitor 100 to be aware of a likely heat stroke approaching.

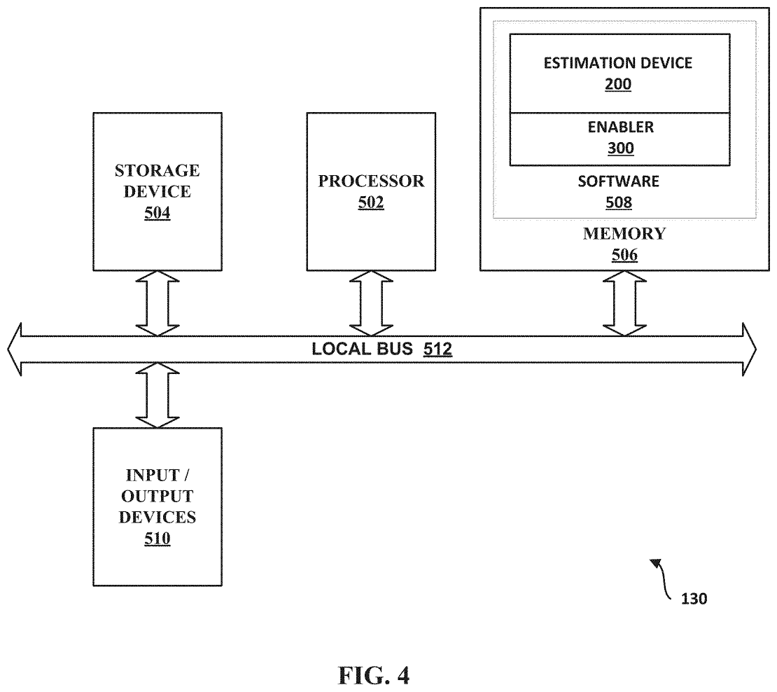

FIG. 4 is a schematic diagram illustrating embodiments in which the estimation device 200 functionality and the enabler 300 functionality are provided within a computer 130. The computer 130 contains a processor 502, a storage device 504, a memory 506 having software 508 stored therein that defines the abovementioned functionality, input and output (I/O) devices 510 (or peripherals), and a local bus, or local interface 512 allowing for communication within the computer 130. The software 508 includes the estimate device 200 and the enabler 300. The local interface 512 can be, for example but not limited to, one or more buses or other wired or wireless connections, as is known in the art. The local interface 512 may have additional elements, which are omitted for simplicity, such as controllers, buffers (caches), drivers, repeaters, and receivers, to enable communications. Further, the local interface 512 may include address, control, and/or data connections to enable appropriate communications among the aforementioned components.

The processor 502 is a hardware device for executing software, particularly that stored in the memory 506. The processor 502 can be any custom made or commercially available single core or multi-core processor, a central processing unit (CPU), an auxiliary processor among several processors associated with the present computer 130, a semiconductor based microprocessor (in the form of a microchip or chip set), a macroprocessor, or generally any device for executing software instructions.

The memory 506 can include any one or combination of volatile memory elements (e.g., random access memory (RAM, such as DRAM, SRAM, SDRAM, etc.)) and nonvolatile memory elements (e.g., ROM, hard drive, tape, CDROM, etc.). Moreover, the memory 506 may incorporate electronic, magnetic, optical, and/or other types of storage media. Note that the memory 506 can have a distributed architecture, where various components are situated remotely from one another, but can be accessed by the processor 502.

The software 508 defines functionality performed by the heat response monitor 100, in accordance with the present invention. As previously mentioned, the software 508 in the memory 506 may include one or more separate programs, each of which contains an ordered listing of executable instructions for implementing logical functions of the heat response monitor 100, as described herein. The memory 506 may also contain an operating system (O/S) 520. The operating system essentially controls the execution of programs within the computer 130 and provides scheduling, input-output control, file and data management, memory management, and communication control and related services.

The I/O devices 510 may include input devices, for example but not limited to, a microphone, touch screen, etc. Furthermore, the I/O devices 510 may also include output devices, for example but not limited to, an audio output, a vibration module, a display, LED, or other means of light display, etc. Finally, the I/O devices 510 may further include devices that communicate via both inputs and outputs, for instance but not limited to, a modulator/demodulator (modem; for accessing another device, system, or network), a radio frequency (RF) or other transceiver, or other device.

When the computer 130 is in operation, the processor 502 is configured to execute the software 508 stored within the memory 506, to communicate data to and from the memory 506, and to generally control operations of the computer 130 pursuant to the software 508, as explained above.

When the functionality of the computer 130 is in operation, the processor 502 is configured to execute the software 508 stored within the memory 506, to communicate data to and from the memory 506, and to generally control operations of the computer 130 pursuant to the software 508. The operating system 520 is read by the processor 502, perhaps buffered within the processor 502, and then executed.

When the estimation device 200 and enabler 300 are implemented in software 508, it should be noted that instructions for implementing each of them can be stored on any computer-readable medium for use by or in connection with any computer-related device, system, or method. Such a computer-readable medium may, in some embodiments, correspond to either or both the memory 506 or the storage device 504. In the context of this document, a computer-readable medium is an electronic, magnetic, optical, or other physical device or means that can contain or store a computer program for use by or in connection with a computer-related device, system, or method. Instructions for implementing the estimation device 200 and enabler 300 can be embodied in any computer-readable medium for use by or in connection with the processor or other such instruction execution system, apparatus, or device. Although the processor 502 has been mentioned by way of example, such instruction execution system, apparatus, or device may, in some embodiments, be any computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the processor or other such instruction execution system, apparatus, or device.

Such a computer-readable medium can be, for example but not limited to, an electronic, magnetic, optical, electromagnetic, infrared, or semiconductor system, apparatus, device, or propagation medium. More specific examples (a nonexhaustive list) of the computer-readable medium would include the following: an electrical connection (electronic) having one or more wires, a portable computer diskette (magnetic), a random access memory (RAM) (electronic), a read-only memory (ROM) (electronic), an erasable programmable read-only memory (EPROM, EEPROM, or Flash memory) (electronic), an optical fiber (optical), and a portable compact disc read-only memory (CDROM) (optical). Note that the computer-readable medium could even be paper or another suitable medium upon which the program is printed, as the program can be electronically captured, via for instance optical scanning of the paper or other medium, then compiled, interpreted or otherwise processed in a suitable manner if necessary, and then stored in a computer memory.

In an alternative embodiment, where the estimation device 200 and enabler 300 are implemented in hardware, they can be implemented with any or a combination of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

Optionally, more than one of the heat response monitors may take their findings and transmit them to a central location. An example might be a central office or simply another device which is capable of receiving transmissions from multiple heat response monitors. By providing this environment, the central location is capable of monitoring health of multiple individuals at a time and even further notifying the wearer of the single device if he or she is in risk of heat stroke. In addition, emergency responders or other medical personnel could be notified by those at the central location, or the individual heat response monitors could provide a direct notification to emergency responders or other medical personnel. Further, each heat response monitor may contain an output for the individual wearing of the monitor to be notified of the danger of heat stroke. Examples may include vibration, audible notification, or visual notification.

In accordance with a second exemplary embodiment of the invention, the accelerometer 110, and the heart rate sensor 120 may be located in a place separate from the computer having the estimation device 200 and enabler 300 therein. Specifically, the accelerometer 110 and the heart rate sensor 120 may be located within a single unit for obtaining the biological information required from the human body and attached to the body as previously mentioned. The single unit may transmit determined information to the remote computer where functionality as previously mentioned, may be performed. The remote unit may be close or far from the single unit and transmit via use of a basic transceiver or other means of transmission. Alternatively, the remote computer may be hardwired to the single unit. The remote computer may work independently, only receiving from the single unit, or, alternatively, the remote computer may receive from multiple single units and be used to perform the previously mentioned functionality for each single unit. Results of calculations may be individually transmitted to each user of a single unit, or transmitted to a central device for analysis and notification of necessary parties as previously mentioned. By providing this environment, the central location is capable of monitoring health of multiple individuals at a time and even further notifying the wearer of the single device of the first exemplary embodiment and the wearer of the single device of the second exemplary embodiment of the invention if he or she is in immediate danger of a heat stroke. In addition, emergency responders or other medical personnel could be notified by those at the central location.

We claim:

1. A heat response monitor, comprising:
an accelerometer;
a core temperature sensor;
an estimation device; and
an enabler,
wherein the estimation device uses accelerometry-based functionality to provide a gait-based heat stroke risk score, and wherein the estimation device uses an estimated core temperature of a wearer of the core temperature sensor, to provide an estimated core temperature-based heat stroke risk score, and wherein the gait-based heat stroke risk score and the estimated core temperature-based heat stroke risk score are used to determine if a wearer of the heat response monitor is in risk of heat injury.

2. The heat response monitor of claim 1, wherein the core temperature sensor is a heart rate sensor, which determines the estimated core temperature.

3. The heat response monitor of claim 1, wherein the estimation device detects individual steps in time domain from accelerometry as data is received from the accelerometer.

4. The heat response monitor of claim 1, wherein the estimation device detects individual steps in time domain from accelerometry, as data received from the accelerometer, referred to as accelerometry data, and wherein the estimation device classifies each step in time as a walking or running step.

5. The heat response monitor of claim 4, wherein accelerometry data consists of a time series of 3-axis accelerations, x(t)={x_1(t),x_2(t),x_3(t)} from the accelerometer, with x_1=vertical, x_2=longitudinal, and x_3=horizontal.

6. The heat response monitor of claim 1, wherein the accelerometer and the core temperature sensor are located in different modules.

7. The heat response monitor of claim 1, wherein the accelerometer and the core temperature sensor are located in the same module.

8. A method for predicting exertional heat stroke with a worn sensor, comprising the steps of:
detecting individual steps in time domain from accelerometry, as data is received from an accelerometer, where accelerometry data comprises a time series of 3-axis accelerations;
classifying each step as a walking or running step;
classifying frames of steps as walking frames or running frames;
for frames classified as either walking frames or running frames, computing an autocorrelation of time series data in each acceleration axis x(t), y(t), and z(t) of the frame;
computing average pairwise sample distance in each acceleration axis;
computing change in movement variability features, relative to recent history of feature statistics; and
applying a threshold to a fused risk score, which is a combination of accelerometry-based risk score and estimated core temperature based risk score to predict heat injury.

9. The method of claim 8, wherein classification of each step is based on step duration and standard deviation of acceleration magnitude, which is computed over duration of the step.

10. The method of claim 8, wherein the step of detecting individual steps in time domain from accelerometry is performed as data is received from an accelerometer.
